(19) United States
(12) Patent Application Publication
(10) Pub. No.: US 2023/0380469 A1
(43) Pub. Date: Nov. 30, 2023

(54) COMPOSITION CONTAINING FUNCTIONAL BLACK SOYBEAN POWDER BIOCONVERTED BY USING BACILLUS ENZYMES AS ACTIVE INGREDIENT, AND USE THEREOF

(71) Applicant: KOOKMINBIO, CORP., Jeonju-si, Jeollabuk-do (KR)

(72) Inventor: Moon-Hee SUNG, Seoul (KR)

(73) Assignee: KOOKMINBIO, CORP., Jeonju-si, Jeollabuk-do (KR)

(21) Appl. No.: 18/027,621

(22) PCT Filed: Sep. 23, 2021
(86) PCT No.: PCT/KR2021/012972

§ 371 Date: Mar. 21, 2023

(30) Foreign Application Priority Data
Sep. 21, 2020 (KR) 10-2020-0121669
Sep. 17, 2021 (KR) 10-2021-0124767

(51) Int. Cl. A23L 33/135; A61K 35/742; A61K 38/48; A61K 38/45; A61P 25/16

(52) U.S. Cl. CPC A23L 33/135; A61K 35/742; A61K 38/482; A61K 38/45; A61P 25/16; C12Y 203/02002; C12Y 304/21062

(57) ABSTRACT

The present invention relates to a composition containing bioconverted black soybean powder of which various biologically active functions are improved by treating whole soy milk with an enzyme solution derived from Bacillus polyfermenticus strain. The present invention has identified that low-molecular-weight functional amino acids and peptides are formed by treating whole soy milk with a fermentation solution in which various peptide synthetases and lyases derived from a Bacillus polyfermenticus strain are mixed, and that bioconverted black soybean powder has functional effects such as antioxidant activity and behavioral disorder improvement, and thus a composition for alleviating behavioral disorders, containing bioconverted black soybean powder as an active ingredient, is provided.

### TECHNICAL FIELD

The present disclosure relates to a composition including bioconverted black soybean powder as an active ingredient and a use thereof, and more specifically, to a composition including bioconverted black soybean powder of which various physiologically active functions are improved by treating whole soy milk with an enzyme solution derived from a Bacillus polyfermenticus strain.

### BACKGROUND ART

Motor ability is an ability to exercise using muscle strength, and if motor ability is impaired or off balance, it may cause abnormal movement or gait, making normal movement deterred with bad posture accompanied. In addition, due to reduction in the motor ability, the amount of exercise becomes insufficient, which may cause issues such as physical strength deterioration, obesity, hyperlipidemia, and hypertension. Therefore, in order to sustain a normal, healthy life, it is necessary to improve motor ability.

Methods for improving motor ability that are in use include regular exercise, diet control, and supplements for motor ability enhancement. Research on functional supplements for motor ability enhancement is being actively conducted in both East and West. However, most of supplements for motor ability enhancement used in the West have side effects due to caffeine and anabolic steroids. Recently, research to develop functional supplements using natural ingredients with safety guaranteed is actively on the way but is still inadequate, in that research on supplements that effectively boost motor ability with no side effects is required.

Containing proteins, fats, and various functional components that are good for the body, soybeans are ideal food which is nutritionally excellent and very important and essential in the diet. Moreover, in recent years, with newly known physiological functions such as anti-cancer properties and immune enhancement, the nutritional value of soybeans as a functional food is increasing gradually.

Soy milk, one of the major processed foods of soybeans, is a representative processed soybean product with increased soybean protein utility rate and is known as a functional nutritional drink since it is rich in soybean protein, essential amino acids, and essential fatty acids and contains a large amount of minerals such as iron, phosphorus, and potassium as well as physiologically active substances which are functional components such as isoflavones, saponins, and phytic acid. In addition, research has been conducted to improve functionality by producing peptides which have nutritional functions that promote digestion and absorption by breaking down soybean protein by treating soy milk with proteolytic enzymes as well as physiological activities such as blood pressure enhancement, calcium absorption promotion, anti-allergy, and serum cholesterol reduction. However, its efficacy in enhancing motor ability is unclear yet.

### PRIOR-ART DOCUMENT

### Patent Document

Korean Patent Application Publication No. 10-2011-0027247 (published on Mar. 16, 2011)

### DISCLOSURE OF THE INVENTION

### Technical Goals

In order to solve the above problems, an object of the present disclosure is to provide bioconverted black soybean powder with enhanced functionality and a preparation method thereof, and to provide a composition for improving behavioral disorder including bioconverted black soybean powder as an active ingredient, by treating whole soy milk with a fermentation solution in which various degrading enzymes and peptide-synthesizing enzymes derived from a Bacillus polyfermenticus strain are mixed so as to identify that low-molecular-weight functional amino acids and peptides are formed and the bioconverted black soybean powder derives functional effects such as antioxidant activity and behavioral disorder improvement.

### Technical Solutions

The present disclosure provides a composition for bioconversion including a Bacillus polyfermenticus KMU01 (Accession number: KCTC 11751BP) strain, a culture thereof, a fermented product thereof, or a mixture thereof.

In addition, the present disclosure provides a method of preparing bioconverted black soybean powder, including treating whole soy milk with a Bacillus polyfermenticus KMU01 (Accession number: KCTC 11751BP) strain, a culture thereof, a fermented product thereof, or a mixture thereof.

In addition, the present disclosure provides a pharmaceutical composition for preventing or treating behavioral disorder, including whole soy milk that is fermented using a Bacillus polyfermenticus KMU01 (Accession number: KCTC 11751BP) strain, a culture thereof, a fermented product thereof, or a mixture thereof as an active ingredient.

In addition, the present disclosure provides a health functional food composition for preventing or improving behavioral disorder, including whole soy milk that is fermented using a Bacillus polyfermenticus KMU01 (Accession number: KCTC 11751BP) strain, a culture thereof, a fermented product thereof, or a mixture thereof as an active ingredient.

### Advantageous Effects

According to the present disclosure, it is possible to provide bioconverted black soybean powder and a preparation method thereof, by treating whole soy milk with a fermentation solution in which various degrading enzymes and peptide-synthesizing enzymes derived from a Bacillus polyfermenticus strain are mixed so as to identify that low-molecular-weight functional amino acids and peptides are formed and the bioconverted black soybean powder derives functional effects such as antioxidant activity and behavioral disorder improvement.

### BRIEF DESCRIPTION OF DRAWINGS

FIG. 1 shows a genetic map of a Bacillus polyfermenticus KMU01 strain and genes of an enzyme group.

FIG. 2 shows results of determining degrees of soybean protein degradation and soybean peptide production of whole soy milk and bioconverted black soybean powder.

FIG. 3 is a result of evaluating an antioxidant activity of whole soy milk and bioconverted black soybean powder.

FIG. 4 is a result of evaluating a behavioral disorder improvement effect of whole soy milk and bioconverted black soybean powder.

FIG. 5 is results of evaluating a behavioral disorder improvement effect of whole soy milk and bioconverted black soybean powder under oxidative stress.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

As such, as a result of endeavor to develop a functional composition including bioconverted black soybean powder with an excellent behavior disorder improvement function, the present inventors completed the present disclosure by identifying an excellent behavior disorder improvement effect of a composition including the bioconverted black soybean powder obtained by hydrolyzing whole soy milk using an enzyme group in which various proteolytic enzymes and peptide-synthesizing enzymes that are secreted and produced by GRAS fermented food-derived microorganisms isolated from fermented food are mixed.

The present disclosure provides a composition for bioconversion including a Bacillus polyfermenticus KMU01 (Accession number: KCTC 11751BP) strain, a culture thereof, a fermented product thereof, or a mixture thereof.

The bioconversion refers to a technology for converting an existing material (substrate) using a biological reaction of microorganisms or enzymes produced by microorganisms, specifically, the bioconversion refers to conversion of soybeans or black soybeans (substrates) using an enzyme group which is a culture supernatant in which various enzymes produced by the Bacillus polyfermenticus KMU01 (Accession number: KCTC 11751BP) strain are included, and products obtained by the bioconversion is referred to as bioconverted black soybean powder.

The Bacillus polyfermenticus KMU01 (Accession number: KCTC 11751BP) strain was registered in Korean Collection for Type Cultures (KCTC) on Aug. 25, 2010 under the name of Bacillus amyloliquefaciens Kimchi, but was later renamed as Bacillus polyfermenticus KMU01 on Jun. 27, 2018 as the precise species name thereof was identified as Bacillus polyfermenticus.

The culture may be an artificial medium obtained by culturing the Bacillus polyfermenticus KMU01 (Accession number: KCTC 11751BP) strain, and the fermented product may be a natural medium fermented using the Bacillus polyfermenticus KMU01 (Accession number: KCTC 11751BP) strain.

The artificial medium may be a commercially produced synthetic medium capable of culturing Bacillus polyfermenticus or bacteria, such as tryptic soy broth (TBS), tryptic soy broth (TSB) nutrient broth (NB), and Luria-Bertani broth (LB), but is not limited thereto.

The natural medium refers to a natural product that may be fermented by bacteria and may be a medium in which natural products such as potatoes, tomatoes, and milk are used, but is not limited thereto.

The culture and the fermented product may exhibit activities of protease, gamma-glutamyltransferase (GGT), and nattokinase.

The protease is an enzyme that hydrolyzes peptide bonds between amino acids constituting a protein with the proteolytic enzyme, some of which include exopeptidases that cleave amino terminus (aminopeptidase) or carboxy terminus (carboxypeptidase) of a protein as well as endopeptidases (e.g., trypsin, chymotrypsin, pepsin, papain, elastase) that cleave the middle of a protein. The gamma-glutamyltransferase (GGT) is an enzyme that transfers the glutamyl group in a gamma-glutamyl compound to a suitable receptor (amine) and is a type of transacylases. The nattokinase, a thrombolytic enzyme produced by Bacillus natto during growth by ingesting nutrients of soybeans when fermenting soybeans, includes vitamin B complex and a large amount of antioxidant enzymes.

In addition, the present disclosure provides a method of preparing bioconverted black soybean powder, including treating whole soy milk with a Bacillus polyfermenticus KMU01 (Accession number: KCTC 11751BP) strain, a culture thereof, a fermented product thereof, or a mixture thereof.

The bioconverted black soybean powder as used herein refers to a powder form of a product obtained by carrying out reactions between black soybean, the fruit of a perennial vine of the legume family, and microorganisms or enzymes produced by microorganisms so as to cleave or degrade carbohydrates and proteins contained in the black soybean, specifically the bioconverted black soybean powder refers to powder of products obtained by converting the black soybean using an enzyme group which is a culture supernatant in which various enzymes produced by the Bacillus polyfermenticus KMU01 (Accession number: KCTC 11751BP) strain are included.

The black soybean refers to the fruit of a legume such as soybeans, white beans, black turtle beans, kidney beans, and peas, and is not limited thereto the type of fruit of the legume plant.

The whole soy milk may be treated with the Bacillus polyfermenticus KMU01 (Accession number: KCTC 11751BP) strain, culture thereof, fermented product thereof, or mixture thereof at a concentration of 3% (v/v) to 7% (v/v), preferably 5% (v/v).

The treatment with the Bacillus polyfermenticus KMU01 (Accession number: KCTC 11751BP) strain, culture thereof, fermented product thereof, or mixture thereof may be performed at 35° C. to 40° C. for 3 to 5 hours, preferably at 37° C. for 4 hours.

The bioconverted black soybean powder may exhibit an antioxidant activity.

In addition, the present disclosure provides a pharmaceutical composition for preventing or treating behavioral disorder, including whole soy milk that is fermented using a to Bacillus polyfermenticus KMU01 (Accession number: KCTC 11751BP) strain, a culture thereof, a fermented product thereof, or a mixture thereof as an active ingredient.

The behavioral disorder is a kind of diseases such as reduction in motility and sleep time due to excessive breakdown and lack of dopamine, such as movement disorders, cognitive dysfunction, Parkinson's disease, eating disorders, attention deficit disorders, and sleep disorders.

The pharmaceutical composition of the present disclosure may be prepared in a unit dose form or prepared by infusion in a multi-dose container through formulation using pharmaceutically acceptable carriers according to a method that may be easily carried out by a person skilled in the art to which the present disclosure pertains.

The pharmaceutically acceptable carriers are those commonly used in preparation and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and, mineral oil, but are not limited thereto. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional preparation.

The pharmaceutical composition may be formulated in the form of one or more external preparations selected from the group consisting of injectable formulations such as aqueous solutions, suspensions, and emulsions, pills, capsules, granules, tablets, creams, gels, patches, sprays, ointments, emplastrum agents, lotions, liniments, pastas, and cataplasmas.

The pharmaceutical composition of the present disclosure may include pharmaceutically acceptable carriers and diluents, which are additional for formulation. The pharmaceutically acceptable carrier and diluent include excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol, but are not limited thereto. The pharmaceutically acceptable carrier and diluent may be biologically and physiologically compatible with subjects. Examples of the diluent may include saline, aqueous buffers, solvents, and/or dispersion media, but are not limited thereto.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, powder preparation, granules, emulsions, hard capsules, soft capsules, syrups, or elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the degree of diseases, administration duration of a composition, administration methods, administration periods or intervals, excretion rate, and drug forms, and may be appropriately selected by those skilled in the art. For example, the dosage may be in the range of about 0.1 to 10,000 mg/kg but is not limited to thereto, and it may be administrated in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on formulation methods, administration methods, administration duration, and/or administration routes of the pharmaceutical composition, and those skilled in the art may easily determine and prescribe the dosage effective for desired treatment. Administration of the pharmaceutical composition of the present disclosure may be conducted once a day or several times in divided doses.

In addition, the present disclosure provides a health functional food composition for preventing or improving behavioral disorder, including whole soy milk that is fermented using a Bacillus polyfermenticus KMU01 (Accession number: KCTC 11751BP) strain, a culture thereof, a fermented product thereof, or a mixture thereof as an active ingredient.

The present disclosure may be generally used as a commonly used food product.

The food composition of the present disclosure may be used as a health functional food. The term “health functional food” as used herein refers to food manufactured and processed with raw materials or components having useful functionality for the human body in accordance with the Health Functional Food Act, and the term “functionality” as used herein refers to the intake to derive effectiveness in health care such as regulation of nutrients or physiological actions for the structure and function of the human body.

The food composition of the present disclosure may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills.

For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying disease by administering the composition according to the present disclosure. The term “treatment” as used herein refers to any action that improves or favorably changes the symptoms of a disease by administering the composition according to the present disclosure. The term “improvement” as used herein refers to any action that improves the bad state of a disease by having an individual intake or administer the composition of the present disclosure.

### MODES FOR CARRYING OUT THE INVENTION

Hereinafter, example embodiments will be described in detail to help the understanding of the present disclosure. However, the following example embodiments are merely illustrative of the content of the present disclosure, and the scope of the present disclosure is not limited to the following example embodiments. The example embodiments of the present disclosure are provided to more completely explain the present disclosure to those of ordinary skill in the art.

### Example 1. Evaluation on Various Activities of Enzymes Derived from Fermented Food Microorganisms

Various enzymatic activities of a Bacillus polyfermenticus KMU01 (Accession number: KCTC 11751BP) strain, a fermentation strain, were evaluated to prepare functional bioconverted black soybean powder.

First, activities of protease, gamma-glutamyltransferase (GGT), and nattokinase were evaluated. The fermentation strain was inoculated in 50 mL of tryptic soy broth (TSB) medium and cultured at 37° C. for 24 hours, and a supernatant of the culture was collected and centrifuged at 8,000 rpm for 20 minutes. Supernatants of centrifuged culture were used to evaluate each enzymatic activity.

For the activity of protease, 0.1 ml of 0.5% azocasein solution and 0.1 ml of coenzyme solution were added to an eppendorf tube as substrates, a reaction was performed in a constant temperature water bath at 37° C. for 1 hour, and then 0.4 ml of 10% trichloroacetic acid solution was added to stop the reaction. The reaction solution was centrifuged at 13,000 rpm for 5 minutes to collect the supernatant which was then neutralized by adding 0.6 ml of 0.525 N NaOH solution to 0.6 ml of the supernatant, the absorbance was measured at 420 nm, and the activity of protease was evaluated by setting, as 1 unit, an amount of enzymes that free 1 μg of tyrosine for 1 minute under the reaction condition.

For the activity of gamma-glutamyltransferase (GGT), 0.01 ml of coenzyme solution and 0.09 ml of 50 mM phosphate buffer solution (pH 7.0) containing 0.1 mM r-L-glutamyl-p-nitroaniline (p-NA-Glu, Sigma-Aldrich) were mixed, a reaction was performed at 40° C. for 30 minutes, and then 0.01 ml of 3.5 N acetic acid was added to stop the reaction. The amount of free p-nitroaniline was measured at 410 nm. Using p-nitroaniline as a standard solution, the enzymatic activity was calculated by drawing a standard curve. For 1 unit of enzymatic activity of GGT, a degree of enzymatic activity of GGT was evaluated by calculating the amount of enzyme that frees 1 mole of p-nitroaniline from p-NA-Glu per minute.

For the activity of nattokinase, 350 μl of 50 mM borate buffer (pH 8.5), 100 μl of 1% fibrinogen solution, and 25 μl of 10 unit thrombin solution were mixed, a reaction was performed at 37° C. for 10 minutes, and then 25 μl of coenzyme solution was added, followed by a reaction at 37° C. for 1 hour. 500 μl of 0.2 M TCA solution was added to the reaction solution to stop the reaction, and then the mixture was allowed to stand at 37° C. for 10 minutes. After collecting the supernatant by centrifuging the reaction solution at 8,000 rpm for 20 minutes, the absorbance of the collected supernatant was measured at 275 nm, and the enzymatic activity was calculated according to the following calculation formula to evaluate a degree of the thrombolytic activity.

Degree of thrombolytic activity (FU/ml)=A1−A0/0.01×1/60×1/0.025×D

A1: Absorbance value of a sample

A0: Absorbance value (blank) of a blank test sample prepared without addition of coenzyme solution 0.01: Activity of enzymes with absorbance increased by 0.01 per minute

60: Enzyme reaction time (min)

0.025: Amount of enzymes used

D: Dilution rate of a sample

As shown in Table 1 below, protease activity was found to be 78 U/ml, GGT activity was 3500 mU/ml, and nattokinase activity indicating the hemolytic activity was 24 U/ml.

TABLE 1

| Evaluation on enzymatic activity | B. polyfermenticus KMU01 |
|---|---|
| Protease activity (U/ml) | 78 |
| GGT activity (mU/ml) | 3500 |
| Nattokinase activity (U/ml) | 24 |

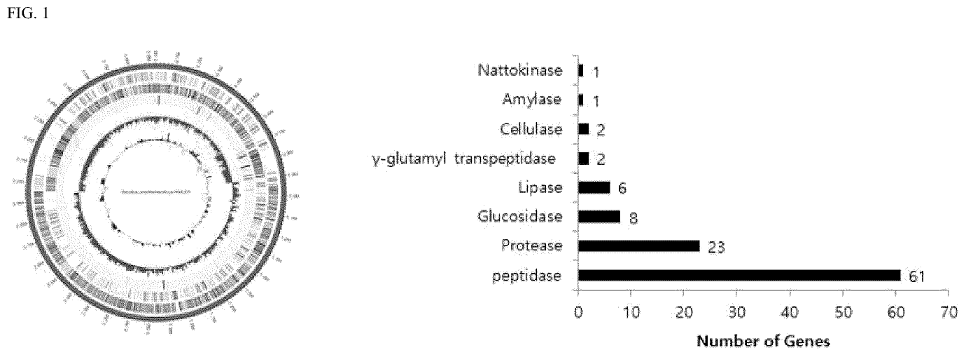

In addition, the genome of the Bacillus polyfermenticus KMU01 (Accession number: KCTC 11751BP) strain was analyzed with PacBio_20K sequencer and SMRT 2.3.0 (HGAP2) assembler to identify genes of various functional enzymes. As a result, as shown in FIG. 1, it was determined that the Bacillus polyfermenticus KMU01 (Accession number: KCTC 11751BP) strain has 61 peptidase genes, 23 protease genes, 8 glucosidase genes, 6 lipase genes, 2 r-glutamyl transpeptidase (GGT) genes, 2 cellulase genes, amylase genes, and nattokinase genes.

### Example 2. Preparation of Functional Bioconverted Powder Using the Fermentation Strain

Used as an enzyme solution for bioconverting whole soy milk was a supernatant obtained by culturing the Bacillus polyfermenticus KMU01 (Accession number: KCTC 11751BP) strain in tryptic soy broth (TSB) medium at 37° C. for 24 hours. In order to prepare the whole soy milk, cv. Socheongja from Iksan was washed and immersed in water for 14 hours, and then the water was removed, followed by grinding using a grinder while removing water. The ground sample was boiled at 100° C. for 30 minutes, and then whole soy milk was obtained. The obtained whole soy milk was treated with the enzyme solution at a rate of 5% (v/v), a reaction was performed at 37° C. for 4 hours for bioconversion, and then lyophilization was carried out to prepare the bioconverted black soybean powder.

TABLE 2

| Classification | Conditions for bioconversion |
|---|---|
| Whole soy milk (mL) | 95 |
| Enzyme solution (mL) | 5 |
| Reaction temperature (° C.) | 37 |
| Response time (hr) | 4 |

### Example 3. Evaluation on a Degree of Hydrolysis of the Bioconverted Black Soybean Powder

A degree of hydrolysis of the bioconverted black soybean powder prepared in Example 2 for proteins was evaluated. 2 mL of hydrolysates from each sample were taken, placed in a test tube containing 2 mL of 20% (w/v) trichloroacetic acid (TCA), and then centrifuged (3,000λg, 10 min) after mixing, and a certain amount of centrifuged supernatant was taken to measure an amount of protein and calculate the degree of hydrolysis. As a result of calculation, it was found that the degree of hydrolysis of the bioconverted black soybean powder was 53.8%.

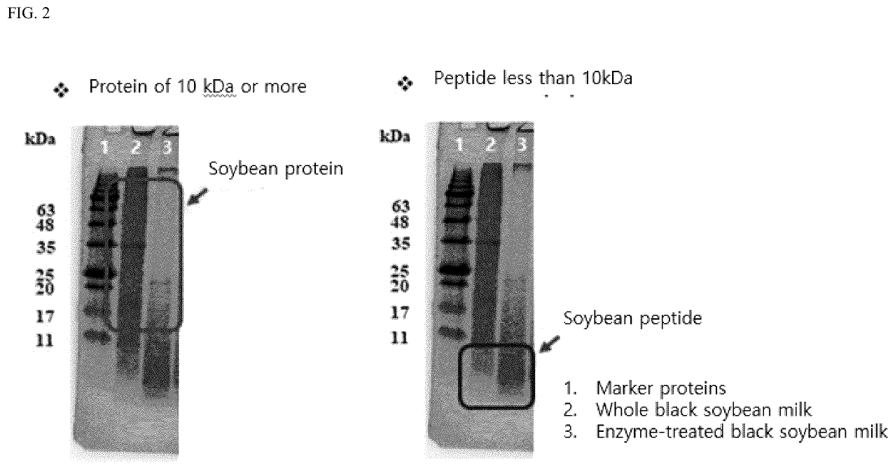

In addition, as a result of identifying a difference in molecular weight of the black soybean protein by performing 10% sodium dodecyl sulfate polyacrylamide gel electrophoresis (SDS-PAGE), as shown in FIG. 2, compared to a control group (whole black soybean milk), the content of soybean peptides of 10,000 Da or less was increased 1.23 times in the bioconverted black soybean powder (enzyme-treated black soybean milk).

### Example 4. Analysis of a Composition of Amino Acids in the Bioconverted Black Soybean Powder

An automatic amino acid analyzer (Biochrom 30+) was used to analyze functional amino acids in the bioconverted black soybean powder. As shown in Table 3 below, the content of functional amino acids such as branched chain amino acids (BCAAs) required for muscle growth and aromatic amino acids which are precursor amino acids of neurotransmitters has been increased.

TABLE 3

| | | Content (mg/L, mg/soybean 70 g) | |
|---|---|---|---|
| | | Whole soy milk | Bioconverted black |
| Classification | Amino acid | (Control group) | soybean powder |
| Aromatic | Phe | 8 | 65 |
| | Tyr | 5 | 28 |
| | Trp | 14 | — |
| BCAA | Val | 8 | 72 |
| | Leu | 3 | 65 |
| | Ile | 1 | 21 |
| Others | GABA | 2 | 12 |
| | Glu | 17 | 9 |
| | Cys | 5 | 14 |
| | His | 8 | 75 |
| | Pro | N/D | 51 |
| | Lys | 3 | 79 |
| | Arg | 110 | 111 |

### Example 5. Evaluation on an Antioxidant Activity of the Bioconverted Black Soybean Powder

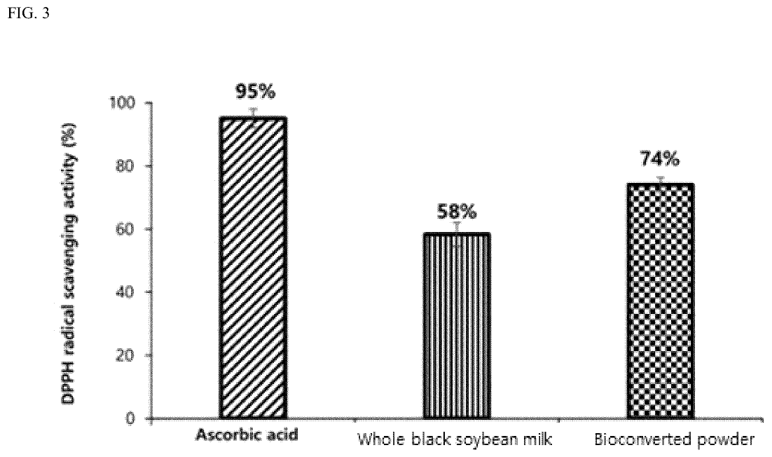

DPPH radical scavenging activity was analyzed to evaluate an antioxidant activity of the bioconverted black soybean powder. For DPPH radical scavenging ability, as a method of measuring, using a spectrophotometer, a degree of reduction in DPPH radical by reacting 1,1-diphenyl-2-picrylhydrazyl (DPPH), a stable free radical, with a predetermined sample solution, 50 μl of the sample and 50 μl of 0.1 mM DPPH solution were mixed, the mixture was allowed to stand in a dark room at room temperature for 30 minutes, and then the absorbance was measured at 517 nm to calculate the degree of radical reduction compared to the control group. Blank absorbance was measured by mixing 50 μl of water and 50 μl of 0.1 mM DPPH solution, and the control absorbance of each sample was measured by mixing 50 μl of the sample and 95% ethanol. As a sample of a positive control group, ascorbic acid was used. As shown in FIG. 3, high DPPH radical scavenging activity of 74% was shown in the bioconverted black soybean powder compared to the control group (whole black soybean milk).

### Example 6. Evaluation on a Behavioral Disorder Improvement Effect of the Bioconverted Black Soybean Powder

In order to observe whether the bioconverted black soybean powder has a functional effect such as behavioral disorder improvement, fruit flies were orally administered with the control group (whole black soybean milk) and the bioconverted black soybean powder (enzyme-treated black soybean milk) for 5 weeks to evaluate the change in behavior differences in each fed group and non-fed group via climbing assay.

Since fruit flies have a habit called negative geotaxis, they are used in many behavioral studies. Negative geotaxis is a term to express a behavior of defying gravity after dropping fruit flies to the ground through application of a single impact to a glass tube and a test tube that include fruit flies, and is often used in the climbing assay. Parkinson-model fruit flies have negative geotaxis but shows lower negative geotaxis than normal individuals due to deletion of the DJ-1 gene. The deletion of the DJ-1 gene accelerates a rate of dopamine breakdown, leading to reduction in motility and sleep time, such as symptoms of Parkinson's disease.

The fruit flies used therefor were wild-type fruit flies w¹¹¹⁸ and DJ-1B^{ex54}, Parkinson-model fruit flies, to evaluate the behavioral disorder improvement effect of the bioconverted black soybean powder. The Parkinson-model fruit fly is a model from which the DJ-1 gene is deleted, and the deletion of the DJ-1 gene accelerates a rate of dopamine breakdown to cause phenomena such as reduction in motility and sleep time, such as symptoms of Parkinson's disease. All fruit flies were cultured at 12-hour intervals during day and night with temperature at 25° C. and 60% humidity maintained.

First, in order to observe the behavioral disorder improvement effect using fruit flies, the model animal, according to the feeding of the bioconverted black soybean powder, the climbing assay, which is to analyze the behavior of defying gravity after dropping fruit flies to the ground through application of impact to a test tube in which fruit flies exist, was performed.

The fruit flies were orally administered with the whole soy milk and the bioconverted black soybean powder for 5 weeks, and the climbing assay, the behavioral analysis of fruit flies, was performed in 18 cm-long vials. After adaptation to the environment at room temperature for 10 minutes, the experiment was performed based on a time point at which the fruit flies were completely seated on the ground, and then the number of fruit flies that rose to 8 cm or more from a starting point for 10 seconds was measured, wherein the experiment was repeated 4 times. (n>10) However, T-test was used for statistics, the significance level (P) was set to 0.05, and P<0.05 was determined to be significant.

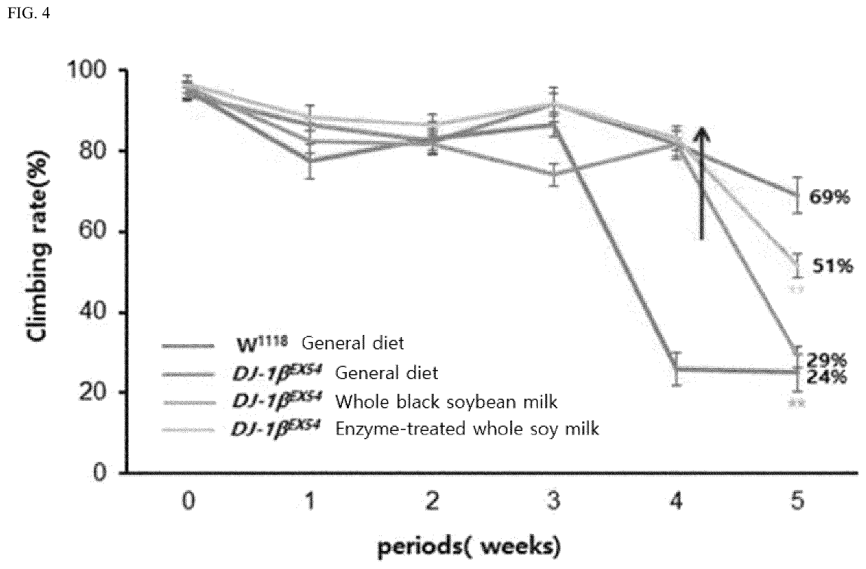

As shown in FIG. 4, climbing ability of the Parkinson's disease fruit fly model (DJ-1B^{EX54}) that was fed with general fruit fly meal (general diet) decreased by 25% at week 4 and then by 24% at week 5, while the Parkinson-model fruit fly (DJ-1B^{EX54}) that was fed with the bioconverted black soybean powder (enzyme-treated black soybean milk) showed similar climbing ability to wild fruit flies until week 4 and maintained 51% climbing ability when that of wild fruit flies was 69% at week 5, thereby proving that the bioconverted black soybean powder had the behavioral disorder improvement effect.

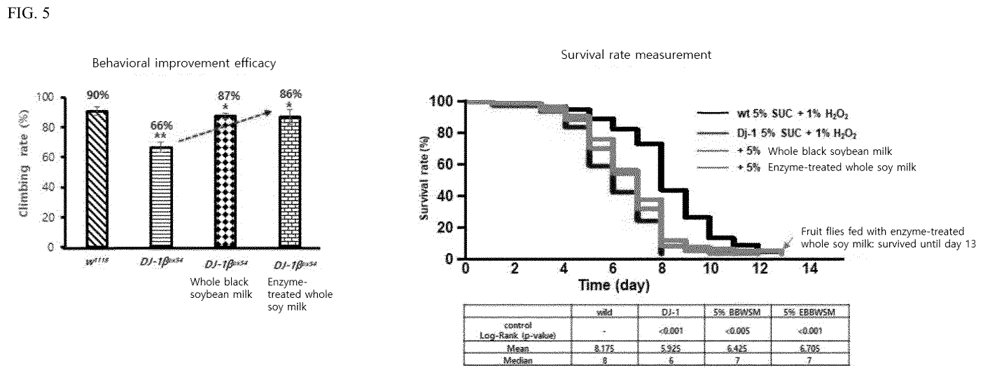

In addition, in order to find whether the bioconverted black soybean powder feeding under artificial oxidative stress is effective in improving the behavioral disorder of fruit flies, 5% sucrose feed containing 1% H₂O₂ and 5% sucrose feed containing 5% bioconverted black soybean powder (enzyme-treated black soybean milk) and 1% H₂O₂ were orally administered to fruit fly models for 13 days to check climbing assay and survival rates. As shown in FIG. 5, Parkinson-model fruit flies (DJ-1B^{EX54}) under the artificial oxidative stress showed significantly low climbing rates and survival rates, while DJ-1B^{ex54} fruit flies fed with the whole black soybean milk and the bioconverted black soybean powder (enzyme-treated black soybean milk) showed a high climbing rate compared to the control group, with the highest survival rate observed in a group fed with the bioconverted black soybean powder.

As described above, a specific part of the content of the present disclosure is described in detail, for those of ordinary skill in the art, it is clear that the specific description is only a preferred embodiment, and the scope of the present disclosure is not limited thereby. In other words, the substantial scope of the present disclosure may be defined by the appended claims and their equivalents.

1. A method of preventing or treating behavioral disorder, comprising:
administering a pharmaceutical composition comprising a Bacillus polyfermenticus KMU01 (Accession number: KCTC 11751BP) strain, a culture thereof, a fermented product thereof, or a mixture thereof as an active ingredient to a subject.

2. The method of claim 1, wherein the culture is an artificial medium obtained by culturing the Bacillus polyfermenticus KMU01 (Accession number: KCTC 11751BP) strain.

3. The method of claim 1, wherein the fermented product is a natural medium fermented using the Bacillus polyfermenticus KMU01 (Accession number: KCTC 11751BP) strain.

4. The method of claim 1, wherein the culture and the fermented product have activities of protease, gamma-glutamyltransferase (GGT), and nattokinase.

5. A method of preparing bioconverted black soybean powder, comprising treating whole soy milk with a Bacillus polyfermenticus KMU01 (Accession number: KCTC 11751BP) strain, a culture thereof, a fermented product thereof, or a mixture thereof.

6. The method of claim 5, wherein treating with the Bacillus polyfermenticus KMU01 (Accession number: KCTC 11751BP) strain, culture thereof, fermented product thereof, or mixture thereof is performed at a concentration of 3% (v/v) to 7% (v/v).

7. The method of claim 5, wherein treating with the Bacillus polyfermenticus KMU01 (Accession number: KCTC 11751BP) strain, culture thereof, fermented product thereof, or mixture thereof is performed at 35° C. to 40° C. for 3 to 5 hours.

8. The method of claim 5, wherein the bioconverted black soybean powder exhibits an antioxidant activity.

9. (canceled)

10. A method of preventing or improving behavioral disorder, comprising:
administering a health functional food composition comprising whole soy milk that is fermented using a Bacillus polyfermenticus KMU01 (Accession number: KCTC 11751BP) strain, a culture thereof, a fermented product thereof, or a mixture thereof as an active ingredient to a subject.

11. The composition of claim 10, wherein the culture is an artificial medium obtained by culturing the Bacillus polyfermenticus KMU01 (Accession number: KCTC 11751BP) strain.

12. The composition of claim 10, wherein the fermented product is a natural medium fermented using the Bacillus polyfermenticus KMU01 (Accession number: KCTC 11751BP) strain.

13. The composition of claim 10, wherein the culture and the fermented product have activities of protease, gamma-glutamyltransferase (GGT), and nattokinase.
